(19) United States
(12) Patent Application Publication
(10) Pub. No.: US 2011/0152263 A1
(43) Pub. Date: Jun. 23, 2011

(54) COMPOSITION AND METHOD FOR INHIBITING NOROVIRUS INFECTION

(76) Inventor: Xi JIANG, Cincinnati, OH (US)

(21) Appl. No.: 11/940,794

(22) Filed: Nov. 15, 2007

Related U.S. Application Data
(60) Provisional application No. 60/866,083, filed on Nov. 16, 2006.

(51) Int. Cl. A61K 31/5377; A61K 31/351; A61K 31/12; A61K 31/17; A61K 31/341; A61K 31/426; A61K 31/381; A61K 31/167; A61K 31/4436; A61K 31/10; A61K 31/415; A61P 31/14

(52) U.S. Cl. 514/236.2; 514/459; 514/685; 514/595; 514/471; 514/680; 514/370; 514/444; 514/617; 514/336; 514/712; 514/406

(57) ABSTRACT

A composition for use in inhibiting the binding of a Norovirus to the histo-blood group antigen on the surface of epithelia. The composition contains a therapeutically effective amount of a binding-inhibiting compound selected from Compounds 1 through 15, and at least one diluent, carrier or excipient. The Compounds competitively bind a Norovirus that has the capability of binding with the histo-blood group antigens of secretor blood type, including A, B, AB, and O blood types. The compositions can be administered to a human prior to or after infection by a Norovirus, to prevent or ameliorate an infection.

### CROSS-REFERENCE TO RELATED APPLICATION

This application claims the benefit of U.S. Provisional Application No. 60/866,083, filed Nov. 16, 2006.

### BACKGROUND OF THE INVENTION

Noroviruses (NV), previously called Norwalk-like viruses, are a leading cause of epidemics of acute, non-bacterial gastroenteritis, affecting people of all ages worldwide. Viruses in this group are spread by fecal-oral pathways, through person-to-person transmission, or by contaminations of environmental surfaces, water or food. The viruses are highly contagious which usually result in large outbreaks in crowded communities or institutions such as schools, restaurants, hospitals, child care centers, nursing homes for the elderly, cruise-ships, and the military settings. Noroviruses are difficult to study because there is no cell culture or animal model available for Noroviruses and the disease is difficult to control because of a lack of vaccine or effective antiviral for Noroviruses. It is therefore a public health priority to develop an effective strategy of prevention and treatment of Norovirus infection.

Noroviruses are small (about 38 nm in diameter), non-enveloped, single-stranded, and positive-sense RNA viruses belonging to the family Caliciviridae. The Norovirus genome encodes three open reading frames (ORF) in which ORF-2 encodes one major structure protein of about 60 kDa that spontaneously forms virus-like particles (VLPs) when expressed in baculovirus or in other expression systems. These VLPs are morphologically and antigenically indistinguishable from the native forms of viruses found in human stools, providing valuable materials for development of immunological assays, for study of virus-host interaction, as a candidate vaccine, and for determination of the structure and capsid assembling of Noroviruses.

Noroviruses are known to recognize human histo-blood group antigens (HBGAs) as receptors. HBGAs are complex carbohydrates linked to glycoproteins or glycolipids that are present on the surfaces of red blood cells and mucosal epithelial cells or as free oligosaccharides in biological fluids such as blood, saliva, milk, and intestinal contents. The HBGA system is controlled by multiple gene families that contain silent alleles, and three major HBGA families, the Lewis, secretor, and ABO families, are involved in Norovirus infection. The recognition of HBGAs by Noroviruses has been found to be highly specific; different Noroviruses recognize different HBGAs, and so far eight distinct receptor-binding patterns have been identified. According to potentially shared antigenic epitopes among different Noroviruses (the A, B, H and Lewis epitopes), the eight binding patterns can be sorted into two groups: the A/B and the Lewis (non-secretor) binding groups. Strains in the A/B binding groups bind to the A and/or B or H epitopes but not the Lewis epitopes, while strains in the Lewis binding group recognize the Lewis and H epitopes but not the A and B epitopes.

The association of HBGAs with Norovirus infection has been demonstrated by human volunteer studies wherein the attachment of Noroviruses to the intestinal epithelium via a matched HBGA receptor is a prerequisite for Norovirus infection. Inhibition of this interaction may result in prevention or control of the viral infection.

The present invention addresses the long felt need for compounds useful in the prevention and treatment of Norovirus infection.

### SUMMARY OF THE INVENTION

The present invention relates to a composition for use in inhibiting the binding of a Norovirus to the histo-blood group antigen on the surface of epithelia, the composition comprising: a therapeutically effective amount of a binding-inhibiting compound selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, and Compound 15, as defined herein, and mixtures thereof, and at least one diluent, carrier or excipient.

The invention further relates to a method for controlling the binding of a Norovirus to a histo-blood group antigen on an intestinal epithelia of a human, comprising the step of administering orally to said human a composition comprising at least one of a binding-inhibiting compound selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, and Compound 15, and mixtures thereof.

The invention also relates to a method for preventing an infection of a mammal by a Norovirus, comprising the step of administering to a mammal an effective preventative amount of a binding-inhibiting compound that inhibits binding of at least one Norovirus to a native histo blood group antigen of the mammal, the compound selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, and Compound 15, and mixtures thereof.

The invention relates also to a method for treating an active infection of a mammal by a Norovirus, comprising the step of administering to the mammal an effective treatment amount of the binding-inhibiting compound that inhibits binding of the infecting Norovirus to the histo blood group antigen of the mammal, the compound selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, and Compound 15, and mixtures thereof.

The binding-inhibiting compound more typically is selected from the group consisting of Compound 1, Compound 2, Compound 4, Compound 5, Compound 10 and Compound 12, and mixtures thereof. The composition and the method provide that the inhibiting compound competitively binds to the Norovirus to prevent or treat an infection by a Norovirus of mammalian host. The composition and method are particularly effective when the human has a secretor blood type, and are particular effective against Norovirus that have a binding pattern against human histo blood group antigens H, A and B.

### BRIEF DESCRIPTION OF THE FIGURES

FIGS. 1A and 1B show the chemical structure of binding-inhibiting compounds 1 through 15.

FIGS. 2A, 2B and 2C show titration curves of the inhibitory activity of the 15 compounds shown in FIGS. 1A and 1B for VA387 virus strain.

### DETAILED DESCRIPTION OF THE INVENTION

Human Noroviruses are believed to replicate and cause disease in the intestinal tract. The attachment through the HBGA receptors on the intestinal epithelia is believed to be a necessary first step of infection, and an inhibition of this step can be an effective treatment of the disease. This treatment may benefit patients who already contracted the infection, and is particularly important for outbreak control of Norovirus gastroenteritis. Administration of a high concentration of an inhibiting compound to all individuals who at risk in an outbreak immediately after the identification of the index patient (prophylactic therapy) provides effective protection and significant control over the outbreak.

Noroviruses require only 10-100 infectious particles to initiate an infection and the viral inoculums may not be high titered under a natural condition because Noroviruses are transmitted by person-to-person contact, or by contaminated surface, food, or water. Thus, it may not be difficult for the inhibiting compounds to compete with the intake virus that might initiate the infection.

The treatment may also reduce the symptoms of a patient even after the onset of disease if the administered compound(s) have sufficient affinity to compete with the virus for the HBGA receptors. Furthermore, if the inhibiting compounds are highly stable and remain functional, they may further reduce the infection by blocking the progeny viruses for subsequent cycles of replication.

The human intestinal tract contains a large surface area and the HBGA receptors are highly abundant on the mucosal surface, making it difficult for a compound to block all receptor binding sites on the surfaces. The human intestinal tract is a complicated environment that contains various components (salts, bile acids, enzymes, digested and undigested food and nutrients) and extreme chemo-physical conditions that could interfere with the compound functioning as an antiviral against Noroviruses. Therefore, in addition to high affinity and high specificity, the inhibiting compounds should survive in the intestinal tract and have good biosafety in the host.

### (a) Compositions

The invention includes a medicament or pharmaceutical composition comprising an active compound selected from a Norovirus binding-inhibiting compound, and a pharmaceutically acceptable diluent, carrier or excipient. Typically the composition comprises at least one binding-inhibiting compound that can prevent a Norovirus from binding with at least one histo-blood group antigen. Typically the composition comprises at least one binding-inhibiting compound that can prevent a Norovirus from binding with an H epitope, and/or with an A epitope, and/or with a B epitope, and/or with a Lewis epitope. An effective composition comprises a plurality of binding-inhibiting compound that can bind with any type of Norovirus regardless of the Norovirus binding pattern to the histo-blood group antigens.

The amount of the binding-inhibiting compound in the composition is typically from about 1,000 to about 100,000 units per dose, where a unit defines the amount of the binding-inhibiting compound to bind with a single Norovirus particle.

Non-limiting examples of suitable pharmaceutically acceptable diluents and carriers include phosphate buffered saline solutions, water, emulsions including oil/water emulsions, various types of wetting agents such as detergents, and sterile solutions. Compositions comprising such carriers can be formulated by well known conventional methods. Compositions can also comprise liquid or viscous compositions that can coat and/or line the surface of the GI tract, thereby placing the active compounds in direct proximity with the epithelial cells.

### (b) Methods of Treatment

The invention relates to a method for preventing an infection of a host, typically a mammal, by a Norovirus, by administering to the host an effective preventative amount of the binding-inhibiting compound that inhibits binding of at least one Norovirus to a native histo blood group antigen of the host.

The invention can also relate to a method for treating an active infection of a host by a Norovirus, by administering to the host an effective treatment amount of the binding-inhibiting compound that inhibits binding of the infecting Norovirus to the histo blood group antigen of the host.

When an outbreak of a Norovirus occurs, the time to isolate and detect the specific strain of Norovirus for pinpoint treatment can delay administration of treatment or prevention compositions to a population of infected or susceptible persons. Preferably, a combination of compounds in a single medicament or pharmaceutical composition that can singularly or jointly bind with any strain of Norovirus, will ensure effective treatment or prevention of infection, regardless of the particular strain(s) of Norovirus involved.

The effective prevention amount of the prevention compound is an amount sufficient to bind most or all of the Norovirus capsids or particles that are present in the gastrointestinal system of a host who had consumed a food, water, or other source contaminated by the Norovirus. Ordinarily, the contaminating amounts of Norovirus would be very low. The amount of the binding-inhibiting compound to be consumed for prevention or treatment typically ranges from about 100 to about 10,000 units per dose, more preferably from about 1,000 to about 10,000 units per dose, where a unit defines the amount of the binding-inhibiting compound to bind with a single virus particle. In one embodiment of the method of the invention, a dose of the medicament comprising the binding-inhibiting compound is consumed by the host just prior to, while, or just after, consuming a food or water suspected of being contaminated with a Norovirus.

The effective treatment amount of the binding-inhibiting compound is an amount sufficient to bind most or all of the Norovirus capsids or particles that are progeny from those infected within the epithelial cells of the gastrointestinal system of the host. Ordinarily, these amounts or levels of Norovirus are high compared to the amount of Norovirus found in the contaminated water or food. The amount of the binding-inhibiting compound to be consumed will typically range from about 1,000 to about 100,000 units per dose, more preferably from about 10,000 to about 100,000 units per dose, where a unit defines the amount of the compound to bind with a single Norovirus particle. In an embodiment of the method of the invention, a dose of the medicament comprising the binding-inhibiting compound is consumed by the host periodically until the symptoms of the infection have dissipated and stopped. Since any consumed compound would pass through the gastrointestinal system in the ordinary course, the periodic dosage is preferably administered about every 1 to 4 hours.

The compounds preferably are non-toxic and have no adverse side effects on the host, including non-binding or associating with other antigens in the host's system. This can be demonstrated based on in vitro experiments.

### (c) Binding Patterns of Noroviruses to Human Histo-Blood Group Antigens

The following Table A shows six known binding patterns found to have been formed by one or more strains of Norovirus, with human histo blood group antigens (HBGA).

TABLE A

| Pattern no. | HBGA(s) | Blood Type | Norovirus strain(s) |
|---|---|---|---|
| 1 | A, B, H | A, B, O; secretor | 387, GrV |
| 2 | A, B | A, B; secretor | MOH, PiV, MxV, HV |
| 3 | A | A; secretor | BUDS |
| 4 | A, H | A, O; secretor | Norwalk Virus, C59 |
| 5 | H, Le | non-secretor | VA207*, Boxer** |
| 6 | Le | non-secretor | OIF |

*binds weakly to non-secretor eptitope
**binds strongly to non-secretor eptitope

Such binding patterns are disclosed in detail in US patent publication 2006-0115846, and in co-pending U.S. application Ser. No. 11/264,992 (attorney docket CHM-007R), the entire disclosures of which are incorporated herein by reference.

### (d) Methods and Materials

(1) Saliva-Based Blocking Assay

A saliva based blocking assay offers several advantages for anti-Norovirus agent screening. First, the assay is a solid phase blocking assay which is simple and suitable for large scale screening. Second, the source of HBGAs used in the assay was from healthy human volunteers, which is likely to represent the natural forms of Norovirus receptors. Third, the assay is highly sensitive and specific. Finally, a panel of saliva samples can be assembled from different individuals representing different blood types, from which the results obtained in primary screenings can be validated.

Under our standard protocol of saliva binding assays, VLPs from most of the strains usually resulted in optical density (OD) values greater than 2.5 to their corresponding antigenic epitopes, which is too high for a sensitive screening. To optimize the blocking assays, we have titrated all reagents used in the assays including the VLPs, the first and second antibodies, the blocking reagent (dried cow milk), and a panel of saliva samples from different donors. The final condition of blocking assays that resulted in OD₄₅₀ about 1.0 in the absence of compounds and a 50% of OD₄₅₀ reduction in the presence of compounds was selected as the cut-off for the primary screening. For comparison of daily results, eight control wells containing everything but compounds were included in each plate, and the assays were considered valid when the daily OD₄₅₀ variations in these wells were less than 20%. Less than 1% of the total screening runs failed in our study. The OD values of the eight blank controls that do not contain compounds and VLPs ranged from 0 to 0.02 with little variation among different saliva samples used.

While developing the screening method, it was noted that the inclusion of blotto (dried cow milk) in the step of incubation of compounds with VLPs, significantly decreased the inhibitory activities of the compounds was observed. Cow milk contains a large amount of variable proteins, lipids and glycans which can easily interfere with the assay particularly when a low concentration of compounds (μM) was used in our screening. To avoid any false-negative results, the step of addition of blotto in the EIA for all screening experiments in this study was removed.

(2) Compound Library

A compound library was obtained for the screening, identified as “The Diversity Screening Set” (Timtec Inc., Newark, Del.), which is a collection of diverse, highly pure, rationally selected, drug-like small molecule compounds ranging from 200 to 850 Daltons. A database of the compound library is available from TimTec LLC, Newark, Del. (http://www.timtec.com). The compounds were received as powders which were dissolved in dimethyl sulfoxide (DMSO) to a stock concentration of 10 mM and stored at −20° C. before use. DMSO concentrations in the reaction mixture were managed to be never higher than 0.5% (vol/vol).

(3) Preparation of Norovirus VLPs

Baculovirus-expressed recombinant capsid proteins of four Norovirus strains representing four distinct receptor binding patterns were studied: Norovirus strains Norwalk, VA387, VA207, and MOH. The procedures of production of Norovirus VLPs in insect cell Spodoptera frugiperda (Sf9) have described in “Expression, self-assembly, and antigenicity of the Norwalk virus capsid protein”, J Virol 66:6527-32, Jiang et al (1992), and in “Baculovirus expression and antigenic characterization of the capsid proteins of three Norwalk-like viruses”, Arch Virol 147:119-30, Jiang et al. (2002), the disclosures of which are incorporated herein by reference. Briefly, a cDNA from the 3′ end of the genome containing the viral capsid gene (open reading frame 2 [ORF-2] was cloned from the viral RNA extracted from stool specimens. The recombinant baculoviruses carry the viral capsid genes were constructed from the cloned cDNAs using the Bac to Bac expression system according to the manufacture's instructions (Invitrogen Life Technologies, Carlsbad, Calif.). VLPs were partially purified by sucrose gradient centrifugation and stored at −80° C. Protein concentrations were determined by measuring the optical density at 280 nm (OD₂₈₀) using a GeneQuant spectrophotometer (Pharmacia, Mich.) and by comparison with a bovine serum albumin standard in a sodium dodecyl sulfate-polyacrylamide gel (25). The purity of VLPs used in this study was over 90% which was determined by SDS-PAGE analysis.

(4) Saliva Samples

Saliva samples were collected from healthy adult volunteers under an approval of human subject research protocol by the Institutional Review Board at the Cincinnati Children's Hospital Medical Center. Except for gender and race, no other personal information was collected. A total of 5-10 ml of saliva was collected from each volunteer, and the samples were processed immediately after collection. To avoid potential Norovirus-specific antibodies that may interfere in the receptor binding assays, saliva samples were boiled at 100° C. for 10 min, and then centrifuged at 10,000×g for 5 min. The clear supernatant was stored at −80° C. until use. The HBGA types of the saliva samples were determined by enzyme immune assays with monoclonal antibodies specific to human HBGAs (8). Saliva sample from a type A donor was selected for the primary screening, and a total of 25 saliva samples of known ABO, secretor, and Lewis types were selected for further studies to confirm the primary screening results.

(5) Saliva-Based Enzyme Immune Assay (EIA) to Screen Compounds for Blocking Norovirus Binding to Human HBGA Receptors

The EIA was developed to measure the inhibitory activity of compounds against VA387 VLPs binding to the A antigen. Standard 96-well microtiter plates (Dynex Immulon; Dynatech, Franklin, Mass.) were coated with the type A saliva sample at a dilution of 1:2,000 in phosphate-buffered saline (PBS, PH 7.4). Unbound A antigen was removed by washing with PBS, and the plates were blocked with 5% dried milk (Blotto) for 1 h at 37° C. A total volume of 100 μL of baculovirus-expressed VA387 VLP at a concentration of about 100 ng/ml in PBS were incubated with or without compounds at 37° C. for 30 min, then the reaction mixture was added to the microtiter plates coated with the type A saliva. After 1 hour incubation at 37° C., the bound VLPs were detected using a pooled guinea pig anti-Norovirus antiserum (dilution of 1:3333) obtained by cross-immunization of the animals with 9 different Norovirus strains (8), followed by the addition of horseradish peroxidase (HRP)-conjugated goat anti-guinea pig immunoglobulin G (IgG; ICN, Aurora, Ohio) at a dilution of 1:5000. For each step, the plates were washed 5 times with PBS containing 0.5% Tween 20 (PBS-T). The bound HRP conjugates were detected by the TMB kit (Kirkegard & Perry laboratories) and OD₄₅₀ was read using an EIA spectrum reader (Tecan). The hits from the primary screening were retested with serial dilutions of the compounds (from about 0.1 to about 100 μM), and the blocking activity of each compound was expressed as 50% effective concentration (EC₅₀) by a comparison of the signals in wells with the compounds with that of the negative control wells without compound (column 1) minus the background noise (the blank control wells in column 8 that contain all components except compounds and VLPs).

Following the primary screening, compounds with significant blocking activities against VA387 binding to the A antigen were further tested for their inhibitory activity of: 1) VA387 binding to types B and H antigens, 2) Norwalk virus binding to the A and H antigens, 3) MOH binding to the A and B antigens, and 4) VA207 binding to the Lewis antigens. The same format and condition of the assays described above, except variable concentrations of VLPs (100 to 500 ng/ml), were used. The is because the receptor binding affinity of individual strains varies (8) and based on titration results, we selected a concentration to give an OD₄₅₀=about 1 in the wells without compounds for each strain.

(6) MTS Cytotoxicity Assay

Human cervical carcinoma cells (HeLa) were grown in a humidified 5% CO₂ incubator at 37° C. in RPMI 1640 medium supplemented with 1% L-glutamine, 10% FBS, 100 units/ml penicillin and 100 μg/ml streptomycin (2). Human colon carcinoma cells (Caco-2) were grown in Earle's minimum essential medium (MEM), supplemented with 10% FBS, 1% L-glutamine, MEM nonessential amino acids, N-2-hydroxyethylpiperazine-N9-2-ethanesulfonic acid (HEPES) buffer, penicillin, and streptomycin. The cells were seeded at 5×10⁴ cells per ml onto a 96-well plate and incubated overnight before testing. The MTS cytotoxicity assay was performed using the CellTiter 96 Aqueous Non-radioactive Cell Proliferation Kits (Promega, Madison, Wis.). The assay solution contained the tetrazolium compound MTS (3-(4,5-Dimethylthiazole-2-yl)-5-(3-carboxy-methoxyphenyl)-2-(4-sulfophenyl)-2H-tetrazolium, inner salt) and an electron-coupling reagent, phenazine ethosulfate. The cytotoxicity of individual compounds was determined by the decrease of cellular reduction of MTS into the colored product. Briefly, after the incubations with compounds at various concentrations for 3 days, the culture medium was replaced with fresh media with 100 μl/well of MTS/phenazine methosulfate, incubated at 37° C. for 2 hours, and measured with a plate reader at an absorbance of 490 nm. The 50% cell death concentrations (CC₅₀s) were determined as the concentrations of compounds that caused 50% of the cell death compared to control cells cultured without compound. The selective index (SI) was calculated as the values for the CC₅₀ divided by the EC₅₀ which were determined in the saliva blocking assay described above.

As described herein after in the Examples, inhibiting compounds reacted with specific strains but not others or reacted with the same strains but on different antigens, indicating these inhibiting compounds are specific inhibitors. Norovirus strains within receptor binding patterns or receptor binding groups may share antigenic epitopes. Thus, compounds may be selected as type-common or type-specific for particular receptor binding patterns or receptor binding groups based on their relative reactivities to different strains and different antigens. Most compounds found in this study followed the principle of the classification, but we also observed a compound with variable specificities that was not consistent with others. At this stage it is not known if such compound is extremely strain-specific or the opposite, because the mechanism and the targets on the viral capsids of these compounds remain unknown.

Most of the inhibiting compounds were non-toxic to selected cell lines at relatively high concentrations. In addition, in the characterization of the mechanism of compounds blocking Norovirus binding to HBGAs, it was demonstrated that most of the compounds specifically act on the viruses, not on the HBGA receptors. This would seem to indicate that these compounds unlikely directly contact with the human HBGAs, which might cause unnecessary side-effect to the host cells if the HBGA receptors on the cell surfaces are bound by a compound. This is particularly important for compounds with a low affinity in which a highly efficient drug can be designed by increasing the dosage of the compounds.

In addition to biosafety, the stability of compounds in the intestinal tract should be equally important because inhibiting compounds may be able to block all viruses including the inoculum and progeny viruses during the viral infection. The latter may be more difficult to control because they may have a large quantity and persist in the entire course of infection. Thus stability is an important quality to help prevent any new virus particles generated and released into the lumen of intestine before they enter new cells for new cycle of infections.

### EXAMPLES

### Example 1

### Compound Screening Against A Antigen

Since different Norovirus can share the same antigenic epitopes, such as the H epitope by both the A/B binding and the Lewis binding strains, VA387, a first well-characterized Norovirus strain, was selected for a primary screening of inhibitor compounds. VA387 has the broadest spectrum to human HBGAs that recognizes A, B and H epitopes of the human HBGAs. The A antigen was selected for the target for the VA387.

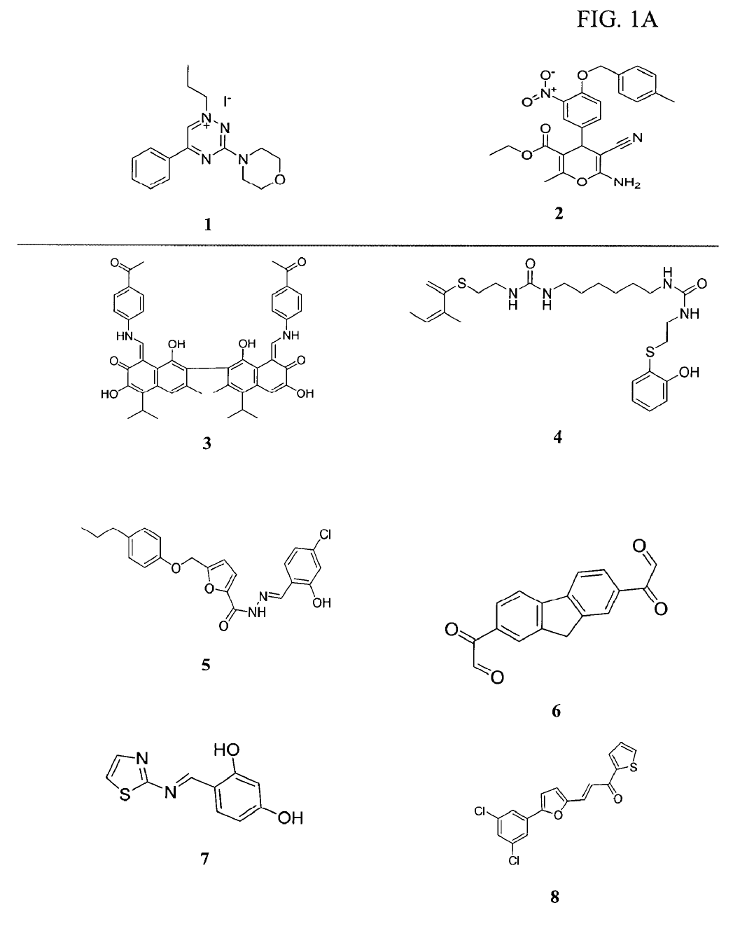

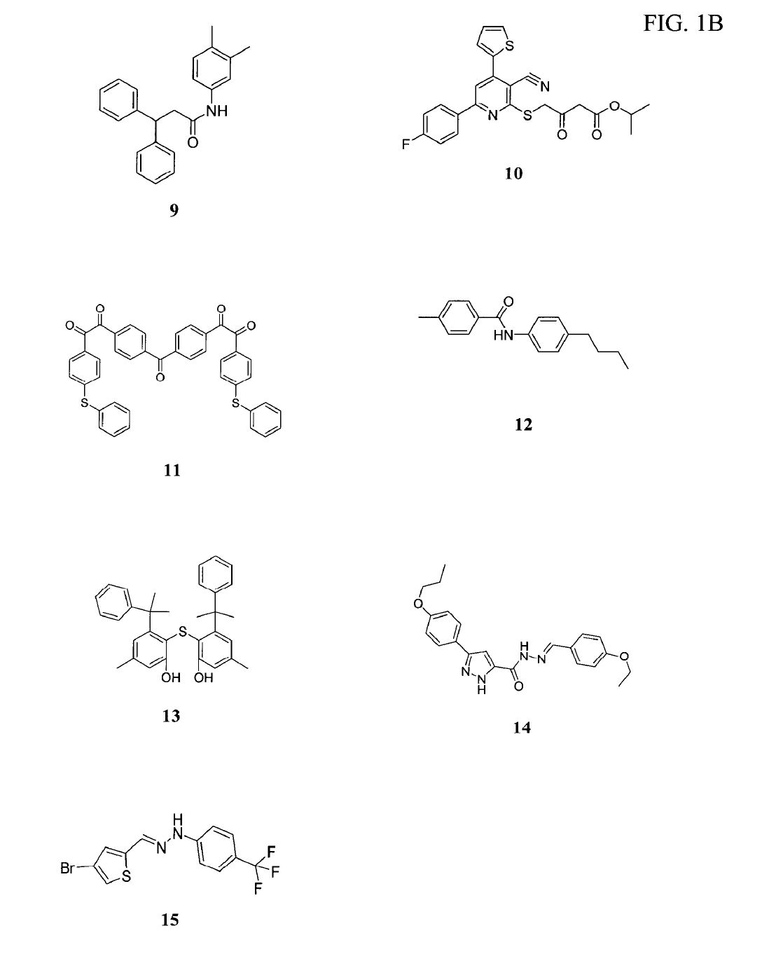

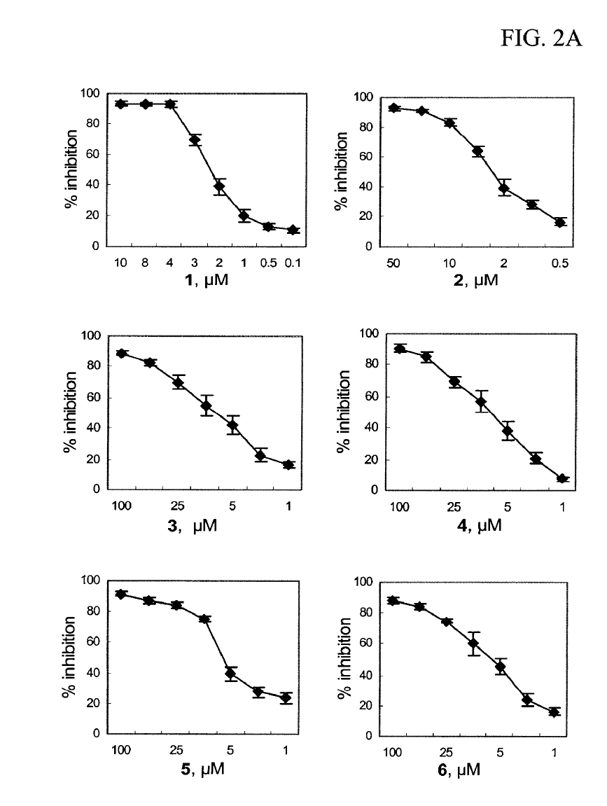

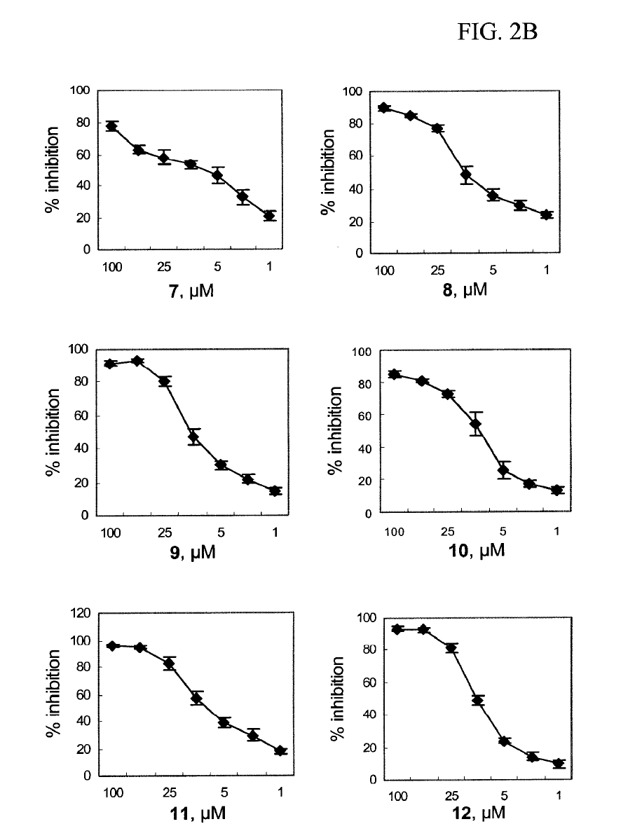

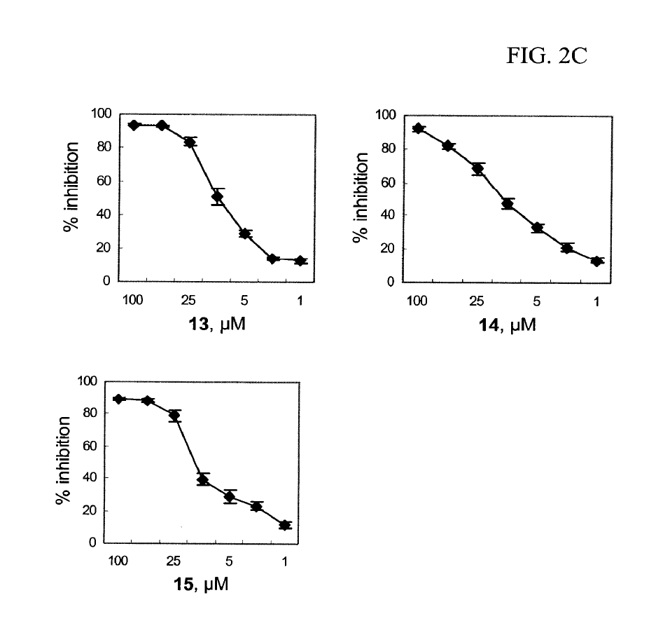

The Saliva-base blocking assay and protocol was used to screen The Diversity Screening Set compound library of about 5000 compounds. Fifteen (15) compounds, numbered 1 through 15 shown in FIGS. 1A and 1B, revealed strong inhibition with EC₅₀ values less than 15 μM against VA387 binding to type A antigen, and showed dose responses at concentrations of about 0.1 to about 100 μM (estimated as 50 μg/ml for any one compound. The most potent inhibitor of binding by VA387 to A antigen was Compound 1, with an EC₅₀ value of 2.2 μM. The titration curves of the inhibitory activity of the 15 Compounds showing inhibiting on VA387 binding to the A antigen are shown in FIGS. 2A, 2B and 2C. The concentrations of the compounds used in the assays were adjusted according to their blocking activities in the primary screening. The concentration ranges for compounds 1 and 2 were from 0.1 to 10 μM, and 0.5 to 50 μM, respectively. The concentration range for compounds 3-15 was 1 to 100 μM. Triplicate tests for each compound were performed and the mean and standard deviation of binding activity reduction are presented. The EC₅₀ of each compound was calculated based on the data presented in this FIGS. 2A, 2B and 2C.

A single dosage of the 15 lead compounds at their EC₅₀s was used to inhibit VA387 binding to a panel of saliva samples from 8 randomly selected type A individuals, and the inhibitory effects of all compounds have been confirmed (data not shown). Significant inhibition was observed for all samples in the presence of compounds, and the reduction rates of the compounds also were comparable to those found in the primary screening. Most compounds had a narrow variation with less than 15% reduction activity differences compared with the primary screening results, indicating the EC₅₀ values determined in the primary screening are highly reproducible.

### Example 2

### Cytotoxicity of Screened Compounds

Two cell lines, the HeLa and Caco-2 cells, were used to evaluate the cytotoxicity of the 15 Compounds using the MTS assay. None of the 15 compounds had significant inhibitory effect on the growth of Caco-2 cells below 200 μM of compound concentration (data not shown). When HeLa cells were used, Compounds 1, 2, 3, 4, 6, 7, 8, 10, 11, 12, and 13 had weak cytotoxicity with CC₅₀ values greater that 50 μM, and only Compounds 14 and 15 showed strong cytotoxicity with CC₅₀ values around 10 μM. The data is shown in Table B, where CC₅₀s stands for the concentrations of compounds to cause 50% cell death. Experiments were repeated three times, and the data in Table B show the average CC₅₀ values.

### Example 3

### Specificity of Inhibitory Activities to the VLP

In preliminary studies to develop the saliva blocking assay, significant blocking activities were observed when compounds and the VLPs were added to the plate at the same time. However, higher blocking activities were observed when the compounds were pre-incubated with the VLPs before addition to the plate. Furthermore, when compounds were added to saliva coated plates as a first step and removed before adding the VLPs, there was no inhibitory activity observed, which was repeatedly observed in testing different VLPs with over 70 active compounds (data not shown). These results suggested that the compounds play their inhibitory role via interaction with the VLPs, not with the HBGAs.

### Example 4

### Screened Compounds Against H and B Antigens

Following the primary screening against the type A antigen, the study was extended for compounds showing some amount of inhibition to determine if the such compounds: 1) inhibit the same Norovirus strain from binding to the B and H antigens, and 2) inhibit other Norovirus strains from the binding to variable antigens. The inhibitory activity of the 15 compounds from Example 1 were then tested on VA387 binding to the B and H epitopes using one saliva sample for each antigen. Compounds 1, 2, 4, 5, 6, 7, 10, 11, 12 and 13 revealed dose-dependant inhibition against the binding of VA387 to both the B antigen and H antigens. Compounds 3, 8, 9, 14 and 15 did not have any inhibitory activity against VA387 binding to the B antigen, and Compounds 3, 8, 14 and 15 did not have any inhibitory activity against VA387 binding to the H antigen, indicating these compounds may act on only the A binding site of the VLP capsid. Compound 9 only revealed inhibitory activity against VA387 binding to the H antigen only, and not to the B antigen. Four of the 10 compounds that blocked both B and H antigen binding revealed strong inhibition (less than 15.0 μM) against both antigens.

These results supported the prediction of different binding sites for different HBGAs on the noroviral capsids.

The overall blocking activities of the 15 compounds were higher to A antigen than B antigen and/or H antigen with one exception (Compound 10), indicating these compounds interact preferentially at the A binding site of the viral capsid.

### Example 5

### Inhibition of Receptor Binding Activities of Variable Strains to Different Receptors

The 15 screened compounds of Example 1 were further evaluated using different Norovirus VLPs representing various HBGA receptor binding types. The prototype Norwalk virus represents a distinct receptor-binding pattern (binding to A and H but not B) from that of VA387. Compounds 1, 2, 4, 5, 10, 12 and 15 blocked Norwalk virus binding to the A antigen, while Compounds 1, 2, 4, 5, 10 and 15 also inhibited Norwalk virus binding to the H antigen. These results indicate a similar binding interface on the VLPs between the two strains.

We also tested a Lewis binding strain (VA207) that is distinct from the AB binding strains. None of the 15 screened compounds had inhibitory activities on VA207 binding, as shown in Table B. This result further supports a classification of the two binding groups of Noroviruses.

Finally, we tested strain MOH that shares the A and B epitope binding (but not H) with VA387. However, only Compound 2 revealed significant inhibitory activity against MOH binding to type A and B salivas with EC₅₀ values of 16.5 and 13.3, respectively. This result was unexpected, and future study is needed to elucidate the difference of the structure and receptor binding interface between the two strains.

TABLE B
Inhibitory effects of the 15 lead compounds against
binding of Noroviruses to the corresponding HBGAs.

| | | | | EC₅₀ (μM) | | | | | |
|---|---|---|---|---|---|---|---|---|---|
| | rVA387 | | | rNorwalk | | rVA207 | rMOH | | |
| Compound | A | B | H | A | H | Le | A | B | CC₅₀ (μM) |
| 1 | 2.2 | 5.8 | 6.8 | 15.6 | 18.9 | >100 | >100 | >100 | 158.0 |
| 2 | 4.8 | 18.6 | 11.9 | 22.9 | 24.5 | >100 | 16.5 | 13.3 | 78.6 |
| 3 | 7.8 | >100 | >100 | >100 | >100 | >100 | >100 | >100 | 82.7 |
| 4 | 8.1 | 13.1 | 14.2 | 14.1 | 22.6 | >100 | >100 | >100 | 112.6 |
| 5 | 9.1 | 13.5 | 7.2 | 18.2 | 36.3 | >100 | >100 | >100 | 46.7 |
| 6 | 9.3 | 44.6 | 41.1 | >100 | >100 | >100 | >100 | >100 | 52.7 |
| 7 | 9.6 | 26.0 | 7.6 | >100 | >100 | >100 | >100 | >100 | 323.6 |
| 8 | 10.9 | >100 | >100 | >100 | >100 | >100 | >100 | >100 | 157.3 |
| 9 | 11.7 | >100 | 47.5 | >100 | >100 | >100 | >100 | >100 | 40.3 |
| 10 | 11.8 | 7.9 | 7.5 | 35.0 | 19.8 | >100 | >100 | >100 | 220.3 |
| 11 | 12.8 | 35.3 | 26.1 | >100 | >100 | >100 | >100 | >100 | 262.8 |
| 12 | 12.8 | 32.5 | 28.2 | 53.0 | 47.8 | >100 | >100 | >100 | 180.2 |
| 13 | 13.6 | 49.5 | 48.1 | >100 | >100 | >100 | >100 | >100 | 70.6 |
| 14 | 13.7 | >100 | >100 | >100 | >100 | >100 | >100 | >100 | 12.3 |
| 15 | 14.1 | >100 | >100 | 22.5 | >100 | >100 | >100 | >100 | 7.6 |

I claim:

1. A composition for use in inhibiting the binding of a Norovirus to the histo-blood group antigen on the surface of epithelia, the composition comprising: a therapeutically effective amount of a binding-inhibiting compound selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, and Compound 15, and mixtures thereof, and at least one diluent, carrier or excipient.

2. The composition according to claim 1 wherein the binding-inhibiting compound is selected from the group consisting of Compound 1, Compound 2, Compound 4, Compound 5, Compound 10 and Compound 12, and mixtures thereof.

3. The composition according to claim 1, wherein the binding-inhibiting compound competitively binds to the Norovirus.

4. The composition according to claim 1 comprising from about 1,000 to about 100,000 units per dose, where a unit defines the amount of the binding-inhibiting compound to bind with a single virus particle.

5. A method for preventing an infection of a mammal by a Norovirus, comprising the step of administering to a mammal an effective preventative amount of a binding-inhibiting compound that inhibits binding of at least one Norovirus to a native histo blood group antigen of the mammal, the compound selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, and Compound 15, and mixtures thereof.

6. A method for treating an active infection of a mammal by a Norovirus, comprising the step of administering to the mammal an effective treatment amount of the binding-inhibiting compound that inhibits binding of the infecting Norovirus to the histo blood group antigen of the mammal, the compound selected from the group consisting of Compound 1, Compound 2, Compound 3, Compound 4, Compound 5, Compound 6, Compound 7, Compound 8, Compound 9, Compound 10, Compound 11, Compound 12, Compound 13, Compound 14, and Compound 15, and mixtures thereof.

7. The method according to claim 5 wherein the mammal is a human having a secretor blood type.

8. The method according to claim 6 wherein the mammal is a human having a secretor blood type.

9. The method according to claim 5 wherein the binding-inhibiting compound is selected from the group consisting of Compound 1, Compound 2, Compound 4, Compound 5, Compound 10 and Compound 12, and mixtures thereof.

10. The method according to claim 6 wherein the binding-inhibiting compound is selected from the group consisting of Compound 1, Compound 2, Compound 4, Compound 5, Compound 10 and Compound 12, and mixtures thereof.
